(19) United States
(12) Patent Application Publication
(10) Pub. No.: US 2023/0028058 A1
(43) Pub. Date: Jan. 26, 2023

(54) NEXT-GENERATION SEQUENCING DIAGNOSTIC PLATFORM AND RELATED METHODS

(71) Applicant: Ohio State Innovation Foundation, Columbus, OH (US)

(72) Inventors: James BLACHLY, Upper Arlington, OH (US); Esko KAUTTO, Columbus, OH (US)

(21) Appl. No.: 17/786,061

(22) PCT Filed: Dec. 16, 2020
(86) PCT No.: PCT/US2020/065193

§ 371 Date: Jun. 16, 2022

Related U.S. Application Data
(60) Provisional application No. 62/948,426, filed on Dec. 16, 2019.

(51) Int. Cl. G16B 20/20; G16B 30/00; G16H 10/60

(52) U.S. Cl. CPC G16B 20/20; G16B 30/00; G16H 10/60

(57) ABSTRACT

A system and method for accurate determination of sequence variants from noisy sequencing data, including single nucleotide variants and structural variants of the internal tandem duplication type. This system expands the utility of inexpensive sequencing instruments which stream relatively high-error output sequences in real time, such that they may be used in high-stakes contexts, such as clinical cancer care. An example application is Acute Myeloid Leukemia (AML), where healthcare providers may need to make decisions in hours, is provided.

### CROSS-REFERENCE TO RELATED APPLICATIONS

This application claims the benefit of U.S. provisional patent application No. 62/948,426, filed on Dec. 16, 2019, and entitled “NEXT-GENERATION SEQUENCING DIAGNOSTIC PLATFORM,” the disclosure of which is expressly incorporated herein by reference in its entirety.

### BACKGROUND

Acute Myeloid Leukemia (AML) is the most incident leukemia in adults. AML is often treated with intensive inpatient chemotherapy, which involves an approximately month-long hospitalization and sometimes results in deaths occurring during this treatment. Failure to rapidly diagnose and treat AML quickly, or treat with the correct therapy, may also be fatal, with a time scale as short as hours to days. Thus, a rapid diagnosis of AML, including its molecular underpinnings, is important. Genetic drivers (and cooperative passengers) have been well understood for decades as providing important diagnostic and prognostic information. Until recently, however, these drivers were un-targetable.

For decades, no new drugs were approved for treating AML. In 2017, the standard of care for newly diagnosed AML in an otherwise fit, younger person was the same as in the mid 1970s. In 1973, Cytarabine plus Daunorubicin (7+3) was published as a method for treating AML and remains the standard front-line treatment. In the late 1970s, bone marrow transplantation was developed and remains a mainstay of treatment. In 2000, Gemtuzumab was approved for treating AML, but was withdrawn as an AML treatment ten years later in 2010.

In the last few years, various targeted therapy treatments have received regulatory approval from the Food and Drug Administration (FDA) for treating AML. In April 2017, Midostaurin was approved by the FDA as an agent for targeting AML having the genetic mutations FLT3-ITD or FLT3-TKD. In August 2017, Enasidenib was approved by the FDA as an agent for targeting IDH2 R140 or R172 mutation to treat AML. In September 2017, Gemtuzumab was re-approved by the FDA as an agent for targeting CD33 to treat AML. In July 2018, Ivosidenib was approved by the FDA as an agent for targeting IDH1 R132 mutation to treat AML. In November 2018: Venetoclax (plus Azacitidine) was approved by the FDA as an agent for targeting BCL2; Glasdegib (plus Cytarabine) was approved by the FDA as an agent for targeting Hedgehog Pathway; and Gilteritinib was approved by the FDA as an agent targeting FLT3-ITD and FLT3-TKD to treat relapsed/refractory AML. Collectively, IDH1 and IDH2 mutations occur in about 20% of AML patients, while FLT3 mutations can occur in up to 30% of the AML patients.

Thus, 2017 and 2018 began a new era in AML treatment. Uptake of new drugs, however, has been limited outside of academic institutions. For historical as well as contemporary logistical reasons, many or most new AML patients are sent to academic medical centers, including leukemia specialty centers. Small-molecule therapies targeting mutant proteins have been rapidly adopted as targeting agents for treating AML. To make use of some targeted small molecule therapies requires diagnostics of a specific genetic lesion (i.e., FLT3 alteration for midostaurin or gilteritinib; and IDH1 or IDH2 mutation for ivosidenib or enasidenib, respectively). The diagnosis of a specific genetic lesion warranting treatment with one of these new agents can be made by using one of the “companion diagnostics” the FDA approved at the same time as each of midostaurin, enasidenib and ivosidenib. However, to consolidate testing and avoid the need to order individual tests for each potentially targetable lesion, many institutions simply use broad next-generation sequencing (NGS) based panels that comprise a dozen to hundreds of genes.

Many targeted therapies, including those that involve the use of ivosidenib, enasidenib, midostaurin, and gilteritinib, cannot be applied without first obtaining a diagnosis of a corresponding targetable mutation. Diagnosis of these mutations by conventional NGS can take more than one week, even at major centers. Furthermore, private oncology offices and smaller hospitals—where much of the cancer care in the United States is delivered—may have only limited (i.e., mail-out) access to these diagnostic tests and simply refer patients to major treatment centers, which further delays the molecular diagnosis. Extra days and weeks of delay in commencing appropriate treatment can be life-threatening and potentially fatal for patients with AML.

Next-generation sequencers are devices that are configured to perform massively parallel DNA sequencing with high throughput. They are capable of rapidly sequencing entire genomes or zooming in to sequence target regions. Large-scale sequencers are of high capital cost and high complexity, and are only typically available at large centers. Recently, small-scale, portable sequencers that can stream output in real-time or finish a run in minutes to hours have become available. Such small-scale, portable NGS instruments hold the potential for reducing the amount of time that is required to perform AML diagnosis and to order a targeted AML treatment to one day or less. In addition, some of these instruments are of relatively low capital cost and could be deployed in resource-constrained settings where conventional NGS is prohibitive. In many cases, however, these small-scale, portable NGS instruments have a detection error rate that is deemed unacceptably high for somatic variant detection. Accordingly, a need exists for a system and method that can leverage the high throughput and ultra-rapid time-to-results of such small-scale, portable NGS instruments while also reducing the detection error rates to acceptable levels.

### SUMMARY

In view of the limitations of cost, time, and complexity in obtaining rapid or real-time determination of nucleic acid sequence variants using conventional sequencing technologies (e.g., second generation sequencing such as sequencing-by-synthesis; Illumina (Solexa) or ThermoFischer Scientific (Ion Torrent)), the present disclosure generally comprises systems and methods for the accurate determination of true variants in noisy sequencing data from newer sequencing instruments such as sequencing biological-nanopore based instruments. The systems and methods described herein strengthen the confidence in variant determination made from noisy sequencing data such as those from biological nanopore-based sequencers.

The general purpose of the systems and methods described herein, described subsequently in greater detail, is to provide high confidence as to the veracity of variants observed in sequencing data from platforms not using conventional sequencing, so that these noisy data may be used in high stakes contexts, such as clinical cancer care.

Because of high error rates compared to conventional technology, platforms not using conventional sequencing have heretofore been mostly overlooked in certain contexts, notably oncology, where low frequency sequence variants may carry great importance, and clinical care implications. The advantage provided by the present systems and methods is that, when coupled with the systems and methods described herein, sequencers of minimal size and cost and with streaming, real-time data output may yield actionable information in minutes, rather than days.

An example computer-implemented method for detecting alleles in a sample is described herein. The method can include receiving a sequencing read, where the sequencing read includes a basecall and a base-wise error score associated with a base within the sequencing read, and receiving a locus-specific error profile for the base, where the locus-specific error profile includes a threshold detection error rate. The method also includes comparing the base-wise error score associated with the base to the threshold detection error rate for the base. The method further includes filtering the base based on the comparison. The base is either accepted as a true variant allele or discarded as a false positive allele based on the comparison.

In some implementations, the base is accepted as the true variant allele when the base-wise error score associated with the base is greater than or equal to the threshold detection error rate for the base. In other implementations, the base is discarded as the false positive allele when the base-wise error score associated with the base is less than the threshold detection error rate for the base.

Alternatively or additionally, the threshold detection error rate is associated with high confidence as to the veracity of variants observed in sequencing data.

Alternatively or additionally, in some implementations, the step of receiving the locus-specific error profile for the base further includes reading the locus-specific error profile for the base from a lookup table (LUT). The LUT stores a plurality of sets of locus-specific error profiles for the base. Additionally, each set of locus-specific error profiles for the base is associated with a different combination of a sequencing device model, a basecaller algorithm, a kit type, and/or a flowcell or chemistry type. Additionally, the sets of locus-specific error profiles for the base are determined by a statistical analysis of the fidelity data from a device that performs basecalling on sequences derived from specimens, the basecalling yielding basecalls and corresponding base-wise error scores.

Alternatively or additionally, the locus-specific error profile is associated with a location of the base in a reference genome. Optionally, the locus-specific error profile is further associated with at least one of a sequencing device model, a basecaller algorithm, a kit type, or a flowcell or chemistry type. The method optionally includes eceiving the at least one of the sequencing device model, the basecaller algorithm, the kit type, or the flowcell or chemistry type associated with the sequencing read.

Alternatively or additionally, the locus-specific error profile is associated with a directionality of basecalling.

Alternatively or additionally, the sequencing read associated with the base is received from a sequencing device. Optionally, the sequencing device is a small-scale next-generation sequencing (NGS) instrument.

Alternatively or additionally, the base is a form of a gene or genomic sequence relevant for diagnosing a disease or condition. Optionally, the disease or condition is Acute Myeloid Leukemia (AML).

An example method of treatment is also described herein. The method includes detecting a true variant allele according to the computer-implemented methods described herein, diagnosing a patient with a disease or condition based upon the detection of the true variant allele, and delivering a therapy to the patient to treat the disease or condition. Optionally, the disease is Acute Myeloid Leukemia (AML).

An example system for detecting alleles in a sample is also described herein. The system includes a processor, and a memory in operable communication with the processor. The memory has computer-executable instructions stored thereon that, when executed by the processor, cause the processor to receive a sequencing read, where the sequencing read includes a basecall and a base-wise error score associated with a base within the sequencing read; receive a locus-specific error profile for the base, where the locus-specific error profile includes a threshold detection error rate; compare the base-wise error score associated with the base to the threshold detection error rate for the base; and filter the base based on the comparison. The base is either accepted as a true variant allele or discarded as a false positive allele based on the comparison.

In some implementations, the system further includes a sequencing device in operable communication with the processor, where the processor receives the sequencing read from the sequencing device. Optionally, the sequencing device is a small-scale next-generation sequencing (NGS) instrument.

In some implementations, the base is accepted as the true variant allele when the base-wise error score associated with the base is greater than or equal to the threshold detection error rate for the base. In other implementations, the base is discarded as the false positive allele when the base-wise error score associated with the base is less than the threshold detection error rate for the base.

Alternatively or additionally, the threshold detection error rate is associated with high confidence as to the veracity of variants observed in sequencing data.

Alternatively or additionally, in some implementations, the step of receiving the locus-specific error profile for the base further includes reading the locus-specific error profile for the base from a lookup table (LUT). Optionally, in some implementations, the processor is configured to maintain the LUT, where the LUT stores a plurality of sets of locus-specific error profiles for the base. Additionally, each set of locus-specific error profiles for the base is associated with a different combination of a sequencing device model, a basecaller algorithm, a kit type, and/or a flowcell or chemistry type. Optionally, in some implementations, the processor is configured to generate the sets of locus-specific error profiles for the base by performing a statistical analysis of the fidelity data from a device that performs basecalling on sequences derived from specimens, the basecalling yielding basecalls and corresponding base-wise error scores.

Alternatively or additionally, the locus-specific error profile is associated with a directionality of basecalling.

Alternatively or additionally, the base is a form of a gene or genomic sequence relevant for diagnosing a disease or condition. Optionally, the disease or condition is Acute Myeloid Leukemia (AML).

Another example system for detecting alleles in a sample is described herein. The system includes a memory device having a plurality of locus-specific error profiles stored at addresses of the memory device, the locus-specific error profiles being based on a device, flowcell and chemistry type, a basecalling algorithm and a kit used to obtain sequencing reads associated with a sample; and a processor in communication with the memory device and being configured to run diagnostic tool, the processor receiving sequencing reads from the device when the device performs the basecalling algorithm to analyze the sample prepared using the kit, and when the processor runs the diagnostic tool, the diagnostic tool performs a filtering algorithm that filters detected alleles by using the locus-specific error profiles to determine whether a detected allele is a real allele or a false positive.

Alternatively or additionally, the device is small-scale next-generation sequencing (NGS) instrument.

Alternatively or additionally, each locus-specific error profile includes a base quality score.

Alternatively or additionally, the processor performs the filtering algorithm by: using a locus at which the detected allele was detected to generate an address in a lookup table (LUT) of the memory device; reading the locus-specific error profile from the address in the LUT; and comparing a base quality score associated with the sequencing reads that contained the detected allele with a base quality score included in the locus-specific error profile read from the LUT to determine whether the detected allele should be treated as a false positive or as a true variant allele.

Alternatively or additionally, if the base quality score associated with the sequencing reads is below a base quality score, X, included in the locus-specific error profile, the detected allele is treated as a false positive, if the base quality score associated with the sequencing reads is equal to or greater than X and less than or equal to Y, where Y is a numerical value that is greater than X, the respective sequencing reads are weighted with a first scalar value that is greater than zero and less than one, and if the base quality score associated with the sequencing reads is equal to or greater than Y, the respective sequencing reads are weighted with a second scalar value that is greater than the first scalar value, the weighting of the respective sequencing reads with the second scalar value causing the detected allele to be treated as a real allele.

Alternatively or additionally, generation of the error profiles in the LUT includes statistical determination of the fidelity data from the device the performs the basecalling algorithm and reports the base and the corresponding base quality score, and the statistical determination originates from sequences derived from specimens with high quality references or truth sets.

Another example method for detecting alleles in a sample is described herein. The method includes using a processor that is configured to run an Acute Myeloid Leukemia (AML) diagnostic tool: receiving sequencing reads from a device when the device performs a basecalling algorithm to analyze the sample, and running the AML diagnostic tool to perform a filtering algorithm that filters detected alleles by using locus-specific error profiles to determine whether a detected allele is a real allele or a false positive, the locus-specific error profiles being stored at addresses of a memory device that is in communication with the processor, each locus-specific error profile being based at least partially on the device, the basecalling algorithm and a kit used to process the sample.

Alternatively or additionally, each locus-specific error profile includes a base quality score.

Alternatively or additionally, the processor performs the filtering algorithm by: using a locus at which the detected allele was detected to generate an address in a lookup table (LUT) of the memory device; reading the locus-specific error profile from the address in the LUT; and comparing a base quality score associated with the sequencing reads that contained the detected allele with a base quality score included in the locus-specific error profile read from the LUT to determine whether the detected allele should be treated as a false positive or as a true variant allele.

Alternatively or additionally, if the base quality score associated with the sequencing reads is below a base quality score, X, included in the locus-specific error profile, the detected allele is treated as a false positive, if the base quality score associated with the sequencing reads is equal to or greater than X and less than or equal to Y, where Y is a numerical value that is greater than X, the respective sequencing reads are weighted with a first scalar value that is greater than zero and less than one, and if the base quality score associated with the sequencing reads is equal to or greater than Y, the respective sequencing reads are weighted with a second scalar value that is greater than the first scalar value, the weighting of the respective sequencing reads with the second scalar value causing the detected allele to be treated as a real allele.

Alternatively or additionally, generation of the error profiles in the LUT includes statistical determination of the fidelity data from the device the performs the basecalling algorithm and reports the base and the corresponding base quality score, and the statistical determination originates from sequences derived from specimens with high quality references.

An example system for detecting structural variants in a sample is described herein. The system includes a processor in communication with the memory device and being configured to run an Acute Myeloid Leukemia (AML) diagnostic tool, the processor receiving sequencing reads from a device when the device performs the basecalling algorithm to analyze the sample prepared using the kit, and wherein when the processor runs the AML diagnostic tool, the AML diagnostic tool performs a detection algorithm to determine whether an internal tandem duplication (ITD) of the FLT3 gene are present in the sample.

Alternatively or additionally, the device is small-scale next-generation sequencing (NGS) instrument.

Alternatively or additionally, the processor performs the detection algorithm by: filtering for reads that meet the criterion of mapping to a locus of interest; filtering for reads that meet the criterion of containing inserted sequence at or above a threshold length N; constructing a distribution of insertion lengths; heuristically selecting one or more peak lengths P={P1,P2, . . . Pn}; selecting from original filtered read set reads containing insertions within a preselected number of nt of identified peaks P and grouping; using a reference sequence, performing consensus calling with peak-specific read groups; for each peak-specific group, updating the reference sequence to incorporate the consensus insertion; and remapping the updated reference sequence to the original reference sequence to derive the final ITD(s).

An example method for detecting structural variants in a sample is also described herein. The method includes using a processor configured to run a diagnostic algorithm that includes receiving sequencing reads from a device when the device performs a basecalling algorithm to analyze a sample; and performing a detection algorithm to determine whether an internal tandem duplication (ITD) of an FLT3 gene are present in the sample.

Alternatively or additionally, the device is small-scale next-generation sequencing (NGS) instrument.

Alternatively or additionally, the processor performs the detection algorithm by: filtering for reads that meet the criterion of mapping to a locus of interest; filtering for reads that meet the criterion of containing inserted sequence at or above a threshold length N; constructing a distribution of insertion lengths; heuristically selecting one or more peak lengths P={P1,P2, . . . Pn}; selecting from original filtered read set reads containing insertions within a preselected number of nt of identified peaks P and grouping; using a reference sequence, performing consensus calling with peak-specific read groups; for each peak-specific group, updating the reference sequence to incorporate the consensus insertion; and remapping the updated reference sequence to the original reference sequence to derive the final ITD(s).

A primary object of the systems and methods described herein is to provide high-confidence single- or multiple-nucleotide variant calls of the substitution, deletion, or insertion type at specific loci using pre-compiled accuracy/error profiles.

An additional object of the systems and methods described herein is to create statistical models of accuracy/error profiles using sequencing reads derived from biological material with ultra-high confidence reference sequences.

An additional object of the systems and methods described herein is to provide high-confidence determination of the presence of one or more structural variants of the Internal Tandem Duplication type.

It should be understood that the above-described subject matter may also be implemented as a computer-controlled apparatus, a computer process, a computing system, or an article of manufacture, such as a computer-readable storage medium.

The details of one or more embodiments of the systems and methods described herein are set forth in the accompanying drawings and the description below. Other features, objects, and advantages of the systems and methods described herein will be apparent from the description and drawings, and from the claims.

### BRIEF DESCRIPTION OF THE DRAWINGS

Many aspects of the systems and methods described herein can be better understood with reference to the following drawings. The components in the drawings are not necessarily to scale, emphasis instead being placed upon clearly illustrating the principles of the present invention. Moreover, in the drawings, like reference numerals designate corresponding parts throughout the several views.

FIG. 1 is a block diagram of the system in accordance with a representative embodiment for performing real-time diagnostics.

FIG. 2 is a flow diagram depicting the method in accordance with a representative embodiment.

FIG. 3 is a flow diagram depicting a filtering process in accordance with a representative embodiment.

FIG. 4 illustrates a process by which the presence, insertion site, sequence, length, and allelic ratio of internal tandem duplication (ITD) in the FLT3 gene can be computed from long-read nanopore data.

FIG. 5 is representative output from the processes depicted in FIG. 4, demonstrating that multiple ITDs can be detected from a single sample.

FIG. 6 is a plot of a representative locus and its error profiles under different models.

FIG. 7 is a flow diagram depicting a method for detecting alleles in a sample according to an implementation described herein.

FIGS. 8A-8B are graphs illustrating the differential error profiles with respect to directionality of basecalling. FIG. 8A is a full-scale version that shows allele fraction (%) versus minimum allele quality for the sense strand (left) and antisense strand (right). FIG. 8B is a limited-scale version of FIG. 8A where Y-axis is limited to 10%.

### DETAILED DESCRIPTION

In accordance with one aspect of the present disclosure, a system and method are provided that leverage the high throughput and rapid time-to-results of certain NGS instruments to perform a diagnosis, such as, for example, an AML molecular diagnosis, very quickly, e.g., within one day or less, while also reducing the effective detection error rate of the NGS. It should be understood that AML molecular diagnosis is provided only as an example application for the systems and methods described herein. This disclosure contemplates that the systems and methods described herein can be used for diagnosis of diseases or genetically-determined conditions other than AML. In accordance with a representative embodiment, a diagnostic tool performs a filtering algorithm that filters each detected allele based on an error profile associated with the position, or locus, of the allele in the genomic sequence to determine whether the detected allele is likely to be a real allele or a false positive. Any detected allele having a variant allele fraction that is at or below the empiric detection capability of the NGS instrument and basecaller for the particular combination of: Q score threshold/library kit/input nucleic acid type/flowcell/basecaller, etc. used is discarded as a likely false positive. Consequently, only detected alleles that have allele fractions that are within the detection capability of the NGS for the particular combination of Q score threshold/library kit/input nucleic acid type/flowcell/basecaller, etc. used are accepted as real by the filtering algorithm. In accordance with another aspect of the present disclosure, the system performs a genomics-based detection method for detecting structural variation in a gene, such as, for example, internal tandem duplication (ITD) in the FLT3 gene.

In the following detailed description, for purposes of explanation and not limitation, exemplary, or representative, embodiments disclosing specific details are set forth in order to provide a thorough understanding of the systems and methods described herein. However, it will be apparent to one of ordinary skill in the art having the benefit of the present disclosure that other embodiments according to the present teachings that are not explicitly described or shown herein are within the scope of the appended claims. Moreover, descriptions of well-known apparatuses and methods may be omitted so as not to obscure the description of the exemplary embodiments. Such methods and apparatuses are clearly within the scope of the present teachings, as will be understood by those of skill in the art. It should also be understood that the word “example,” as used herein, is intended to be non-exclusionary and non-limiting in nature.

The terminology used herein is for purposes of describing particular embodiments only, and is not intended to be limiting. The defined terms are in addition to the technical, scientific, or ordinary meanings of the defined terms as commonly understood and accepted in the relevant context.

The terms “a,” “an” and “the” include both singular and plural referents, unless the context clearly dictates otherwise. Thus, for example, “a device” includes one device and plural devices. The terms “substantial” or “substantially” mean to within acceptable limits or degrees acceptable to those of skill in the art. For example, the term “substantially parallel to” means that a structure or device may not be made perfectly parallel to some other structure or device due to tolerances or imperfections in the process by which the structures or devices are made. The term “approximately” means to within an acceptable limit or amount to one of ordinary skill in the art.

The term “memory” or “memory device,” as those terms are used herein, are intended to denote a non-transitory computer-readable storage medium that is capable of storing computer instructions, or computer code, for execution by one or more processors. References herein to “memory” or “memory device” should be interpreted as one or more memories or memory devices. The memory may, for example, be multiple memories within the same computer system. The memory may also be multiple memories distributed amongst multiple computer systems or computing devices. More specific examples (a nonexhaustive list) of the computer-readable storage medium would include the following: a portable computer diskette (magnetic); solid state memory devices, such as, for example, a random access memory (RAM) (electronic), a read-only memory (ROM) (electronic), an erasable programmable read-only memory (EPROM or Flash memory) (electronic); and optical memory devices, such as, for example, a compact disc read-only memory (CDROM). Note that the computer-readable medium could even be paper or another suitable medium upon which the program is printed, as the program can be electronically captured, via, for instance, optical scanning of the paper or other medium, then compiled, interpreted or otherwise processed in a suitable manner if necessary, and then stored in a computer memory. In addition, the scope of the certain embodiments of the present invention includes embodying the functionality of the preferred embodiments of the present invention in logic embodied in hardware or software-configured mediums.

A “processor” or “processing device,” as those terms are used herein encompass an electronic component that is able to execute a computer program or executable computer instructions. References herein to a system comprising “a processor” or “a processing device” should be interpreted as a system having one or more processors or processing cores. The processor may for instance be a multi-core processor. A processor may also refer to a collection of processors within a single computer system or distributed amongst multiple computer systems. The term “computer,” as that term is used herein, should be interpreted as possibly referring to a single computer or computing device or to a collection or network of computers or computing devices, each comprising a processor or processors. Instructions of a computer program can be performed by a single computer or processor or by multiple processors that may be within the same computer or that may be distributed across multiple computers.

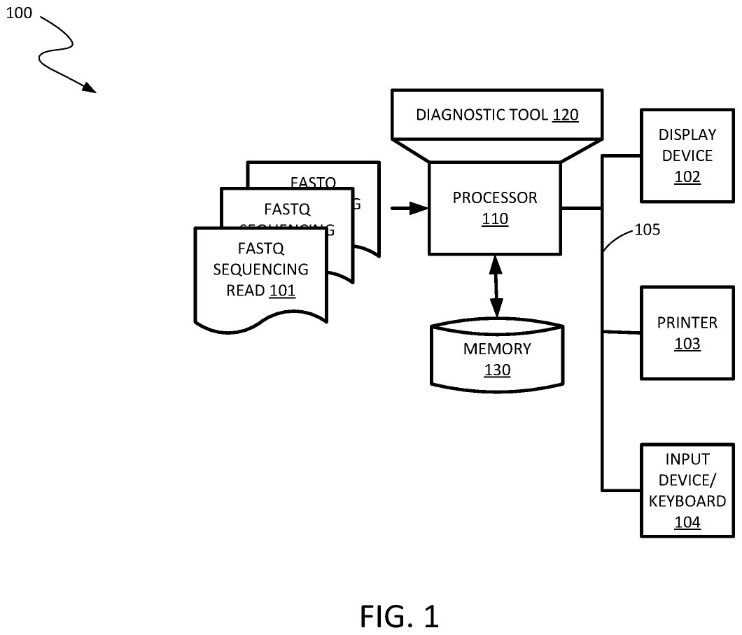

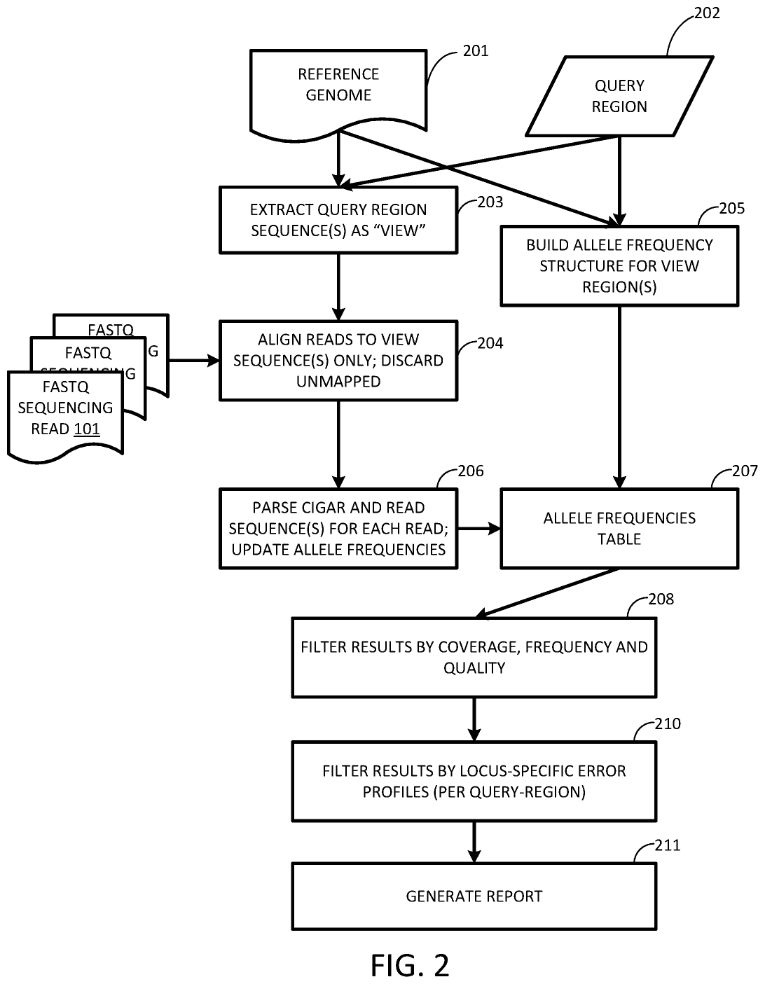

FIG. 1 is a block diagram of the system 100 in accordance with a representative embodiment for performing real-time diagnostic, e.g., including but not limited to AML diagnostics as described herein. The system 100 is configured to receive sequencing reads 101 that are output from an NGS instrument, e.g., a small-scale, portable NGS instrument, such as a MinION MK1b device manufactured by Oxford Nanopore Technologies Ltd. of Oxford, United Kingdom (ONT) and its accompanying basecalling software (which transforms electrical signal to nucleobase sequences). In other implementations, the NGS instrument is a small-scale, non-portable (e.g., benchtop) device, for example, PromethION device manufactured by Oxford Nanopore Technologies Ltd. of Oxford, United Kingdom and its accompanying basecalling software. It should be understood that the NGS instruments manufactured by Oxford Nanopore Technologies Ltd. are provided only as examples. The NGS instrument (not shown) and its accompanying basecalling software outputs sequencing reads 101 that are in a text-based format, such as the FASTQ format. It should be understood that FASTQ format is provided only as an example and that sequencing reads 101 can be output in other formats. A processor 110 of the system 100 receives the sequencing reads 101 and runs a diagnostic tool 120 that processes the sequencing reads 101 in a particular manner described below in more detail with reference to FIG. 2 to obtain a diagnostic result.

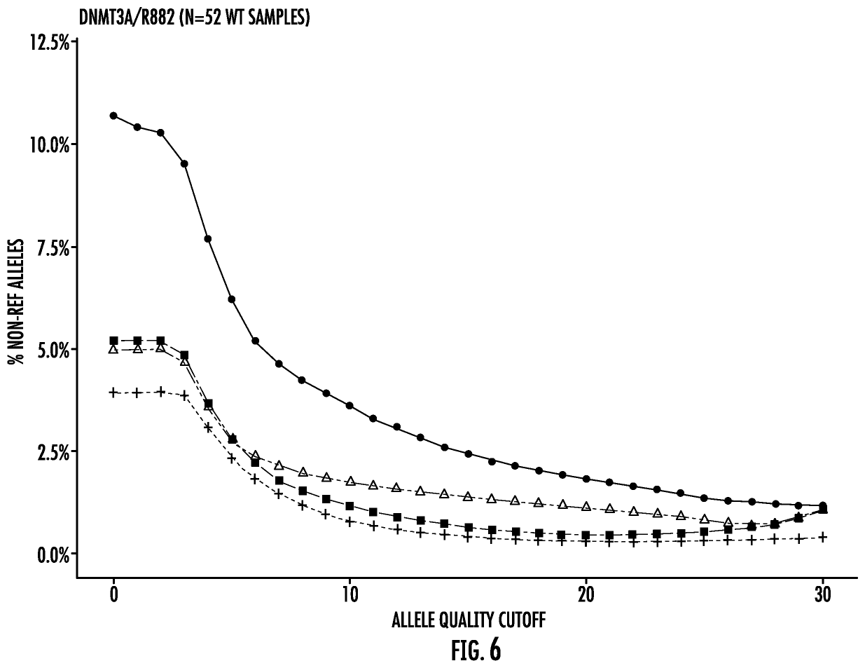

In accordance with a representative embodiment, a memory device 130 of the system 100 contains a lookup table (LUT) that contains error profiles associated with respective loci in the genomic sequence. When an allele is detected, the locus at which it was detected is used by the processor 110 to generate an address in the LUT. The error profile stored at the LUT address corresponds to a detection error rate for detecting alleles at that specific locus for the given NGS and the given combination of Q score threshold/library kit/input nucleic acid type/flowcell/basecaller, etc. being used. When the sequencing reads 101 are generated by the NGS instrument and its accompanying basecalling software, the sequencing reads are output with corresponding base-wise error estimates. These error estimates are referred to as “Q scores” (for quality) or “Phred scores”, and are integers that are related to the (estimated) probability P that the base is mis-called according to the relation Q=−10*log(10)*P. The processor 110 processes the sequencing reads 101 in a manner described below with reference to FIG. 2 to detect potential alleles. When the processor 110 detects a potential variant allele, the processor 110 compares the error rate associated with the sequencing read 101 at the candidate variant allele's position with the detection error rate of the locus-specific error profile obtained from the LUT. If the error rate associated with the sequencing read 101 exceeds the error rate of the error profile (again, for given Q score threshold/library kit/input nucleic acid type/flowcell/basecaller), the detected allele in this read is treated as a false positive. Otherwise, the detected allele is treated as a true variant. The manner in which the error profiles that are stored in the LUT may be generated is described below in detail, for example, by experimentation and statistical analysis with reference to FIG. 6.

The LUT may contain multiple sets of the locus-specific error profiles, with each set corresponding to a particular NGS and a particular combination of Q score threshold/library kit/input nucleic acid type/flowcell/basecaller, etc. Thus, the system 100 may be used to process sequencing reads output from a wide variety of NGS instruments or conditions using a variety of kits/basecallers and remains flexible with run-to-run variation in quality of data produced. The error profiles are determined through systematic experimentation, by determining how often a given NGS instrument [type or model] and combination of conditions correctly detected alleles, and the frequencies of correct and incorrect base calls and the related Q score distributions of these correct and incorrect calls at different genomic loci and in different genomic contexts for different combinations of kits/basecallers, etc.

The system 100 may include an input device 104, such as a keyboard, for example, a display device 102 and/or a printer 103. These devices may be in communication with the processor 110 via one or more buses 105 of the system 100. In accordance with a representative embodiment, the processor 110 causes a human-readable report of the diagnosis to be printed on the printer 103 and/or displayed on the display device 102.

It should be noted that the components of the system 100 are not required to be co-located. For example, the NGS instrument that outputs the sequencing reads 101, the display device 102, the printer 103 and the input device 104 could be at a local site whereas the processor 110, the AML diagnostic tool 120 and the memory device may be components of a data processing center that performs “cloud-based” computing.

FIG. 2 is a flow diagram depicting the method in accordance with a representative embodiment. The method, in accordance with this representative embodiment, comprises the method performed by the diagnostic tool 120 shown in FIG. 1 as well as additional method steps performed by the system 100, some of which are known and performed by existing software solutions that are currently available in the industry running on the processor 110 or on some other processor or computer in communication with processor 110. In accordance with a representative embodiment, blocks 201, 204, 206, 207 and 208 are typical NGS software/workflow steps, block 202 is data dependent on the kit (primer set) used, and blocks 203, 205, 210 and 211 are steps performed by the diagnostic tool 120 in accordance with principles and concepts of the present disclosure. Without limitation, the principles and concepts described herein are also applicable when steps of FIG. 2 are omitted; for example, sequencing reads may instead be mapped to the entire genome (omitting blocks 203, 204).

Block 201 represents the reference genome data structure. Block 202 represents a particular query region being selected based on the chosen kit and primer (or other enrichment technique) set used in the process to obtain the sequencing reads derived from a genetic sample that is loaded into the NGS instrument. In accordance with a representative embodiment, the kit used is a PCR Barcoding Kit, model number SQK-PBK004, by ONT, and the NGS instrument is the aforementioned MinION MK1b NGS from ONT. The FASTQ sequencing reads 101 are produced by basecalling software that converts the raw electrical signal of the NGS instrument into standard text-based format. The processor 110 receives the preselected query region and the reference genome data structure and extracts the query region sequences from the reference genome data structure as the “view,” as indicated by block 203. At block 204, the processor 110 aligns the sequencing reads 101 to the “view” sequences and discards the unmapped sequencing reads. The steps represented by blocks 203 and 204 reduce the amount of data that will need to be processed by the processor 110 in subsequent steps, which increases throughput and decreases processing time. These steps, however, are optional.

At block 205, the processor 110 builds an allele frequency structure for the query region from the reference genome data structure. At block 206, the sequencing reads within the “view” are processed by the processor 110 to identify alleles and to ascertain the frequencies at which the detected alleles occurred. The output of block 204 may be in the form of Sequence Alignment and Mapping (SAM) records (or its binary equivalent, BAM; or its successor CRAM), in which case the process performed at block 206 comprises parsing the records' sequences and Compact Idiosynchratic Gapped Alignment Report (CIGAR) strings to identify and quantify alleles. Block 207 represents the process of an allele frequencies table being generated and updated based on the allele frequency structure received from block 205 and the allele frequencies updates received from block 206. At block 208, the results obtained at block 207 are filtered by coverage, frequency and quality. For example, existing variant calling software for Illumina NGS data removes likely false positives with a variety of heuristics. One examples is coverage: loci with fewer than N reads covering may not be reported. Another example is frequency: variant alleles with a frequency that is less than 5% may not be reported. Another example is quality: e.g., remove reads with average Q score (Phred score) less than Q20.

Block 210 represents one of the processes performed by the diagnostic tool 120 to determine whether a detected allele is a real allele or a false positive. At block 210, the filtered results received from block 208 are further filtered based on the aforementioned locus-specific error profiles stored in the LUT. Block 211 represents the step of generating a report of the results obtained at block 210. This report typically contains patient and/or sample identifying information, quality information on the sequencing run, information on the loci being assayed, the presence (including variant-allele frequency) or absence of specific variant alleles, and information on the data used to make these determinations (for example, that the locus was covered with X number of reads, none of which support a variant). The report may be displayed on the display device 102 and/or printed on the printer 103.

As indicated above, the processor 110 and/or the memory device 130 may not be co-located with the NGS that outputs the sequencing reads 101. For example, the processor 110 and memory device 130 may be part of a cloud-computing environment whereas the NGS may be used at a point of care. The display device 102, the printer 103 and the input device 104 may also be located at the point of care or at some other location that is separate from the location where the processor 110 is located.

The filtering process represented by block 210 reduces the number of false positive alleles and increases confidence in alleles that are determined to be real. Thus, the system 100 leverages the advantages of high-throughput, small-scale, portable NGS instruments while reducing detection error rates. The error detection rate can be further improved by only analyzing hotspots.

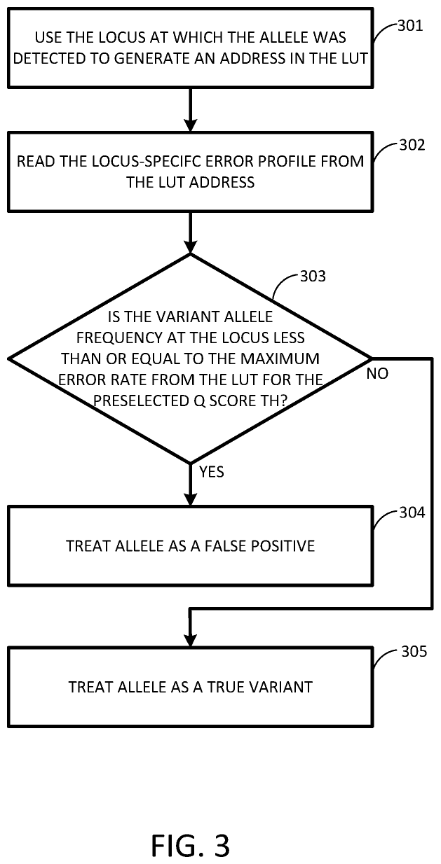

FIG. 3 is a flow diagram depicting the process represented by block 210 in accordance with a representative embodiment. As will be understood by those skilled in the art in view of the description provided herein, there are multiple ways to perform the filter process represented by block 210. The flow diagram of FIG. 3 represents one way to perform the process, but modifications can be made to the process depicted in FIG. 3 without deviating from the inventive principles and concepts. At block 301, the processor 110 uses the locus at which an allele was detected to generate an address in the LUT. At block 302, the processor reads the locus-specific error profile associated with the Q score cutoffs, kit used, flowcell, basecaller, etc. from the address in the LUT. At block 303, the processor determines whether the variant allele frequency associated with the sequencing read that contained the allele is less than or equal to the maximum detection error rate of the locus-specific error profile read from the LUT. If so, the allele is treated as a false positive, as indicated by block 304. If not, the allele is treated as real, as indicated by block 305.

The process depicted in FIG. 3 assumes that the processor 110 receives the particular NGS instrument and the particular kit/flowcell/basecaller that were used and is only accessing portions of the LUT associated with the particular NGS instrument and particular kit/flowcell/basecaller, etc. If there are multiple sets of locus-specific error profiles contained in the LUT, with each set being associated with a particular NGS instrument type and a particular kit/flowcell/basecaller, etc., the processor 110 may generate the LUT address based not only on the locus of the detected allele, but also based on the particular NGS instrument and particular kit/flowcell/basecaller, etc. used.

In the embodiment depicted in FIG. 3, the detection error rate of the error profile that is read from the LUT acts as a threshold (TH) value such that if the variant allele frequency of the sequencing reads is at or below (less than or equal to) the maximum detection error rate of the error profile, the allele is treated as a false positive. For example, if the detection error rate of the error profile is 10% and the variant allele frequency associated with the sequencing reads is less than 10%, the allele is treated as a false positive. An alternative to using the detection error rate as a TH value is to use a TH value that is based on the detection error rate of the error profile plus some factor. For example, if the error detection rate of the error profile is 10%, then the TH value may be 10% plus E, where E is a safety factor. In this example, if ε is 1%, then the TH value would be 11%. It is interesting to note that the adjacent nucleotides may have a vastly different TH.

In the embodiment depicted in FIG. 3, the address in the LUT (Block 302) is dependent also upon the base-wise estimated Q score at the position in the mapped sequencing read corresponding to the locus in question. In this way, multiple error profiles are stored in the LUT, corresponding to different estimates of Q.

As indicated above, another aspect of the present disclosure is a genomics-based detection method for detecting structural variants within a gene, such as the FLT3 gene, for example. The structural variation can be an internal tandem duplication (“ITD”), as a segment of DNA is copied one or more times and pasted adjacent to the original sequence, resulting in a duplication.

The Gold Standard method of detection of an ITD is amplification of the typical ITD-bearing locus (exons 13-15 of FLT3, in that case) with fluorescent primers, and running of the resultant product(s) on a capillary electrophoresis gel via automated instrumentation such as, without limitation, the ABI PRISM Genetic Analyzer. The instrument measures fluorescence as a function of amplicon product size. Detection of a larger fragment than expected is diagnostic of an ITD-bearing locus. An allelic ratio (AR) of ITD to wildtype (wt) is calculated by dividing the area under the curve (AUC) on the capillary electropherogram of ITD peak(s) by AUC of wildtype peak. The AR is used clinically as a cutoff for prognosis and indication for drug treatment. Thus, information obtained from capillary electrophoresis is: presence or absence of ITD, ITD size(s), and allelic ratio. ITD sequence and insertion point are not routinely interrogated, which would require additional sequencing (via Sanger or conventional NGS).

The method and system for detecting an ITD in accordance with the present disclosure provide an alternative to using capillary electrophoresis to detect the ITD while achieving up to 100% sensitivity and 100% specificity compared to capillary electrophoresis data from a clinical laboratory. Capillary electrophoresis is generally not available at the point of care. It is also cumbersome to prepare, requires expensive dedicated equipment, is generally only available at major academic centers or by send-out, and does not reveal all of the information that could be useful (e.g., sequence and insertion point). The method and system for detecting an ITD in accordance with the present disclosure overcome these disadvantages.

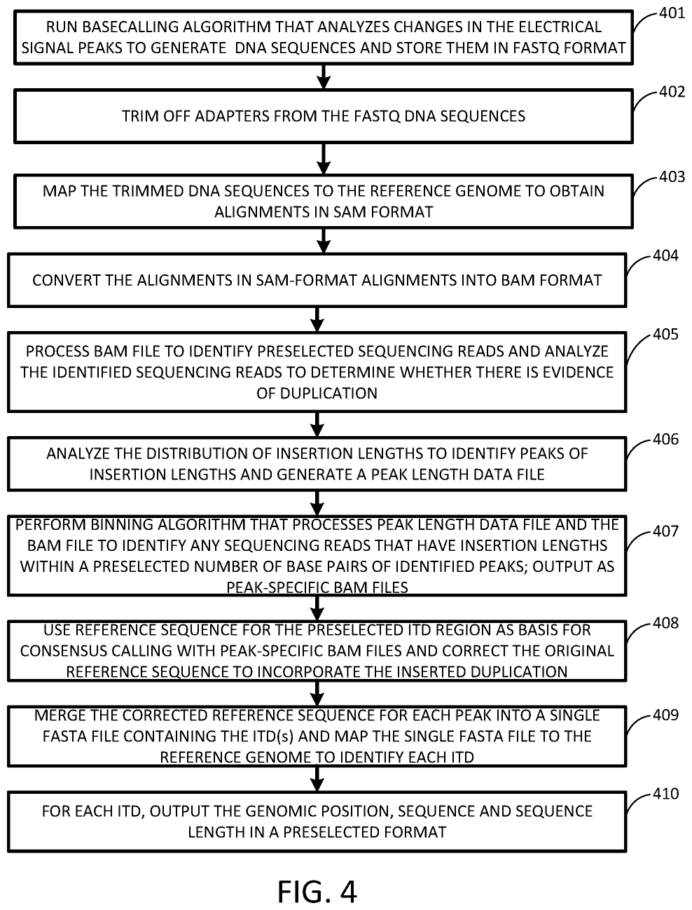

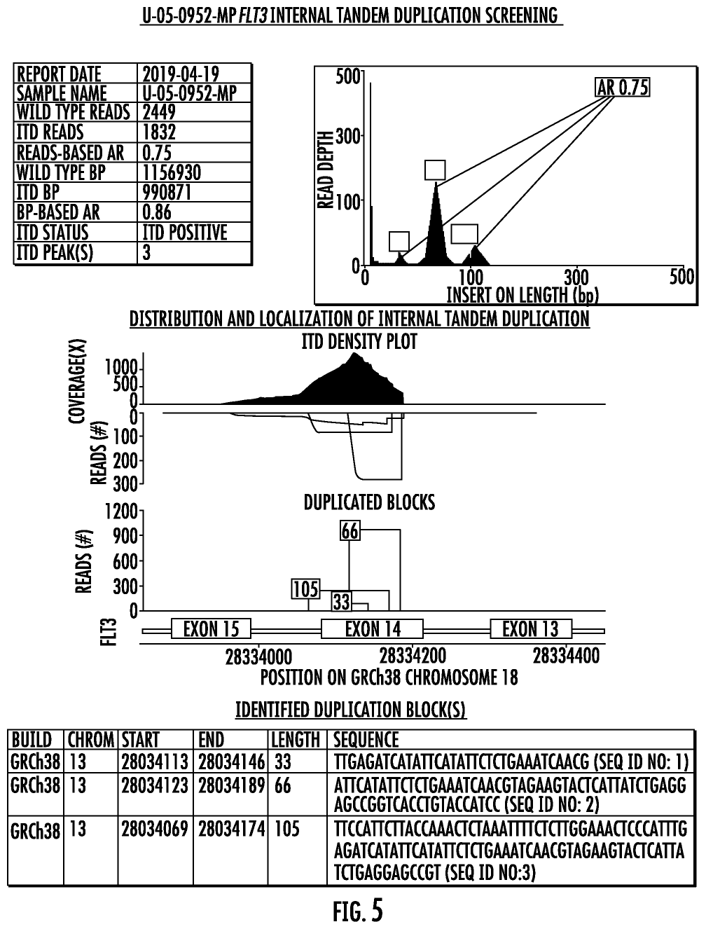

The system 100 shown in FIG. 1 may be used to perform the method. A representative embodiment of the method in which the ONT MinION NGS instrument is used to perform sequencing will now be described with reference to FIGS. 4 and 5. It should be noted, however, that the principles and concepts are not limited to using the ONT MinION NGS instrument or any particular NGS instrument to perform sequencing.

The ONT MinION NGS detects the change in electrical current as a DNA molecule passes through a pore. This is recorded as a set of values that represent the current reading, and signals from one or more DNA molecules (henceforth referred to as “reads”) are stored in FASTS-format files, which are the input to block 401. During the process or step represented by block 401, a base calling algorithm analyzes the electrical signal and extrapolates what the composition of the DNA sequence was based on the changes in electrical signal peaks. The sequence of the DNA is then stored as a sequence of letters (A, T, G, and C) representing the bases that make up the DNA molecule. In accordance with an embodiment, software called Guppy software from ONT that performs a “flip-flop” algorithm was used for this purpose due to its ability to achieve moderately accurate nucleotide sequence deconvolution. These sequences still contain a high rate of false-positive insertions and deletions, confounding routine analysis. The sequences are stored in flat-text files that contain the data for each read, preferably in the FASTQ format.

The detected read sequences will contain the genomic sequence from the adapters used to guide the DNA molecules through the detection wells of the ONT MinION NGS. At the step represented by block 402, a trimming process is performed to detect and remove the adapter sequences from reads, as well as to split any read sequences that may have resulted from two separate molecules being joined by an interjoining adapter. The trimmed data is also recorded out into a FASTQ file. Porechop software was used for this purpose, although the present disclosure is not limited to using any particular software for this purpose.

Next, the read sequences are mapped to a reference or personal genome, which will be assumed, for exemplary purposes to be the GRCh38 human reference genome, as indicated by block 403. In accordance with this embodiment, NGMLR software, which is open-source bioinformatics software, is used for this purpose. This process aligns the reads to that of the reference genome and provides information on what discrepancies (such as changes in nucleotide sequences, or insertions or deletions) there are when compared to the reference genome. The alignments are output in the Sequence Alignment Mapping (SAM) format, which is converted to a binary compressed “BAM” file with the samtools software, as indicated by block 404. The samtools software is open-source bioinformatics software.

The BAM file is then used as input for a data pre-processing algorithm executed by the processor 110 shown in FIG. 1 as part of the diagnostic tool 120. This data preprocessing algorithm is represented in FIG. 4 by block 405. Fundamentally, this algorithm's responsibility within the larger workflow is to categorize reads and compute summary statistics. In the exemplary embodiment, this algorithm identifies which sequencing reads in the data stream originate from exons 13 through 15 of the FLT3 gene and analyzes them to determine which sequencing reads have evidence of duplication of part of the reference genomic sequence. In accordance with this exemplary embodiment, the algorithm considers any reads with insertion of 9 nucleotide (nt) or greater to be potentially ITD-containing, but this value (9) is tunable, i.e., it can vary and is preselected. Any reads that appear to be fragmented or have insufficient mapping quality are discarded from analysis. The number of reads supporting a duplication event above the specified minimum length, and the number of reads without a duplication, are output for the calculation of an allelic ratio (AR) between the alternate (duplicated) sequences and the wild type (normal) sequences. The AR is defined as

$$A R = \frac{\# reads with duplication}{\# reads with out duplication} .$$

The program outputs the data in a plain-text file. Additionally, the length of any duplicated insertion and the genomic start position for the insertion are written out into a separate file for additional processing.

The diagnostic tool 120 also comprises a script that analyzes the distribution of insertion lengths. Block 406 represents running the script. In accordance with an embodiment, the script uses Kernel Density Estimation with a smoothing algorithm to identify “peaks” of insertion lengths. Any peaks that have sufficient support above the background noise level are treated as potential candidate lengths indicating a sequence duplication that is present. The peak length data is written out into a plain text file for further processing. In accordance with an embodiment, an insertion length graph is generated for visual analysis.

The diagnostic tool 120 also comprises a binning algorithm, which is represented in FIG. 4 by block 407. The peak length data and the original BAM file are passed to the binning algorithm, which identifies any reads that have insertion lengths within some number (e.g., +/−5) of base pairs of identified peaks. The reads are then output into peak-specific BAM files, where each distinct peak has supporting reads written into a separate file.

The reference sequence for the ITD region (e.g., exons 13 through 15 of FLT 3) from the reference genome can be stored in a FASTA format file and used as a basis for consensus sequence calling. Racon software, which is open-source software, can be utilized for this purpose, once per peak, with the distinct BAM files and consensus sequence correction being performed on the original (reference) sequence, as indicated by block 408. If sufficient evidence of a conserved insertion sequence is present in the reads, the software “corrects” the original reference sequence and incorporates the inserted duplication into it, writing out another FASTA file.

The FASTA files for each of the detected peaks are merged into a single file containing the sequences for one or more duplication events and the FASTA file containing ITD(s) is then mapped to the reference genome, as indicated by block 409. In accordance with this embodiment, the mapping was performed using the minimap2 algorithm, with the “asm20” preset in this example for assembly-to-assembly alignment. For purposes of this exemplary embodiment, the alignments are output in the PAF format. The diagnostic tool 120 comprises a script that analyzes the pairwise sequence alignments in the PAF file, identifies the exact duplicated sequence, and for each unique ITD outputs the genomic position, sequence, and sequence length into a plain text file, as indicated by block 410.

The output from the previous step and the insertion lengths file from earlier steps are passed to a plotting algorithm of the diagnostic tool 120, which in this example was a program written in the R scripting language. The script preferably generates a graph that shows the localization of each duplicated sequence block, the density of the insertions, and the start positions of the reads. The script aligns the plot to a representation of the ITD region and output as a plot, as shown in FIG. 5. The generated plots, insertion positions and sequences, and allelic ratio data are then used in the construction of a report, an example of which is depicted in FIG. 5, about any potential duplication events that may have been present in the input sequence data.

A representative embodiment for configuring the error profile LUT will now be described. A comprehensive LUT of error profiles at every position in the human genome would in theory consume approximately 24 gigabytes (GB) of RAM ([32-bit float accuracy+32-bit coverage and/or confidence data]×genome size g=3.0×10⁹) for every combination of input type (e.g., native DNA; PCR), pore type, basecaller, and Q score cutoff. This is deemed to be a massively inefficient proposition not conducive to embedded systems, laptop variant-calling, or affordable cloud computing.

The average error rate an example dataset is estimated to be 0.106 (for all Q). Even for sequences of length 10, the accuracies were still remarkably consistent (mean 0.874; median 0.89) with a relatively small number of obvious outliers. Among these outliers were usual suspects (e.g., homopolymers), but also surprising sequences with average GC content and no discernible reason for them to have been basecalled poorly, except rarity in the genome. About 90% of the bottom 1 centile (by accuracy) had obs:exp ratio <1. Likewise, —70% of the top 1 centile had obs:exp ratio >1, i.e., accurate sequences were overrepresented in the genome.

This consistency suggests a strategy whereby an efficient, hybrid data structure can project error rates for the vast majority of sequences probabilistically, while for specific problematic spots or spots of special interest (e.g., cancer-relevant mutational hotspots) storing empirical accuracy data, either from WGS data, targeted sequencing of synthetic references, or both. After computation of genomewide and hotspot kmer accuracy profiles, the error profile LUT was implemented as a hash table with buckets storing loci for which experimental data are available. If the locus is not present in the hash table, its accuracy estimate will be computed from stored kmer-based probabilistic data. This can be structured multidimensionally, encoding as axes relevant experimental variables that the inventors have found to affect error profile substantively. In particular, data for each discrete value of Q can be collected and stored so that downstream consumers may combine reads of many Q into a final statistical estimate of VAF. FIG. 6 is a plot showing error profiles for a representative hotspot (DNMT3A R882) and its dependence on Q.

To analyze the error profiles at each hotspot, the reads from each reference sample were aligned to the GRCh38 human reference genome using (without loss of generality and for demonstrative purposes) the NGMLR aligner. For each hotspot, the samples that were known to be wildtype for the mutation were selected. The 3-base codon for the amino acid in which the mutation occurs were analyzed, storing the called nucleotides at each of the 3 positions and the corresponding base quality scores stored in a multidimensional data frame. It was annotated whether the expected sequence was wildtype (matching the reference), had a mismatch (nucleotide substitution), or an insertion or deletion of nucleotides. The specific mismatch is also recorded. The percentage of reads matching each category at or above each minimum quality score cutoff was calculated to determine the expected overall error rate and error rate of each of the three types (mismatch, insertion, or deletion) of sequencing errors.

Observations indicated that the error profile can be stratified along base quality cutoffs. At or below an average base quality of 5, the error rates for all hotspot mutations are observed to be high. Between the values of 5 and 20, the error rate can be observed to decrease, but still be variable. Above a base quality cutoff of 20, the error rate for most hotspots stabilizes, suggesting a significantly high confidence in the base calls made by the base calling algorithm.

Based on these observations, a scaled variant allele frequency calculation, with reads with an average locus base quality of below 5 being discarded; reads with values in the range of [5,20] being weighted on a scale of [0.0,1.0]; and reads with an average quality of above 20 being treated as a full value of 1, can be used. This allows for lower-quality reads to be included in the variant allele frequency calculations without adding excessive sequencing error and noise to the values.

To further improve accuracy, the process can be repeated separately for mismatches, insertions, and deletions, to identify an optimal scaling for each type of error. Furthermore, mismatches can also be further broken down to represent each potential 3-nucleotide codon that may be incorrectly called, to account for non-random patterns in the error profile. Deletions, likewise, can be calculated as separate error profiles for 1, 2, or 3 base deletions. Insertions can be of any sequence and length, and as such are best treated as a single unit to avoid excessive complexity.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

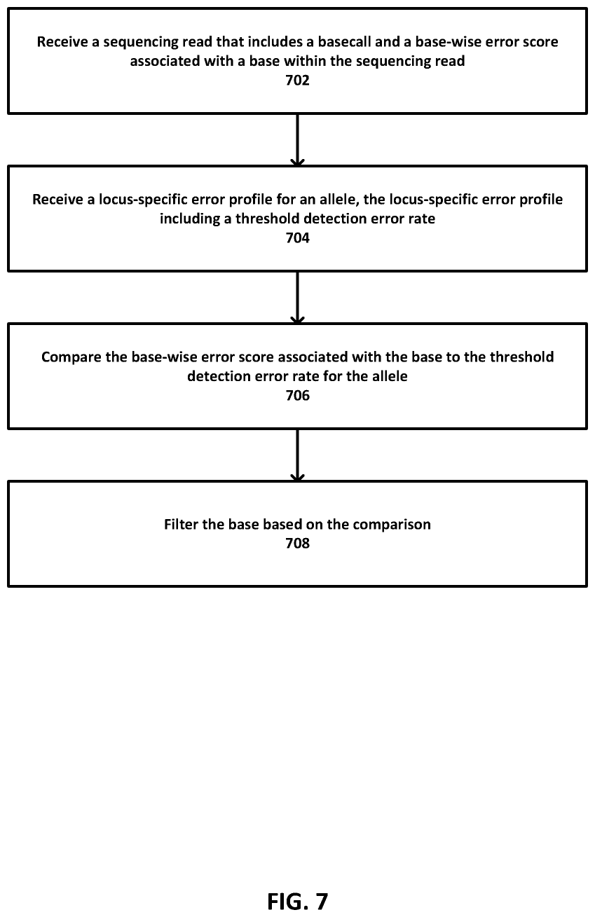

Referring now to FIG. 7, example operations for detecting alleles in a sample are described. This disclosure contemplates that the operations shown in FIG. 7 can be implemented using the system shown in FIG. 1. The method for detecting alleles described with regard to FIG. 7 is capable of accurately determining true variants in noisy sequencing data, for example, sequencing data received from newer sequencing instruments (e.g., NGS instruments). Such NGS instruments are configured to rapidly perform massively parallel DNA sequencing with extremely high throughput. This is particularly desirable for diagnosing diseases such as cancer. However, NGS instruments have a detection error rate that is deemed unacceptably high. As described herein, the operations shown in FIG. 7 address this technical problem by detecting alleles with high confidence as to the veracity of variants observed in sequencing data.

At 702, a sequencing read is received (e.g., by processor 110 of FIG. 1). The sequencing read can be received over one or more communication links from a sequencing device and/or basecalling module. This disclosure contemplates the communication links are any suitable communication link. For example, a communication link may be implemented by any medium that facilitates data exchange including, but not limited to, wired, wireless and optical links. Example communication links include, but are not limited to, a local area network (LAN), a wireless local area network (WLAN), a wide area network (WAN), a metropolitan area network (MAN), Ethernet, the Internet, or any other wired or wireless link such as WiFi, WiMax,3G, 4G, or 5G.

As described herein, a sequencing device such as an NGS instrument reads DNA strands and outputs an electrical waveform. A basecalling module (e.g., hardware, software, or combination thereof) then transforms the electrical waveform into nucleobase sequences. In other words, the basecalling module receives the electrical waveform output by the sequencing device and outputs sequencing reads (e.g., sequencing reads 101 of FIG. 1), which are optionally in text-based format such as FASTQ format, in response. The basecalling module may, in some implementations, use machine learning. Sequencing reads include a series of basecalls and corresponding base-wise error scores (sometimes referred to as quality scores, Q scores, or Phred scores). This disclosure contemplates that the basecalling module is implemented by the sequencing device in some implementations, while in other implementations the basecalling module is implemented by a separate device (e.g., desktop, laptop, tablet, distributed, or cloud computing device(s)). The sequencing read received at step 702 includes a basecall and a base-wise error score associated with a base within the sequencing read. The base within the sequencing read received at step 702 may be a form of a gene or genomic sequence relevant for diagnosing a disease or condition. Optionally, the disease or condition is Acute Myeloid Leukemia (AML) as described in examples herein. Although AML is provided as an example, it should be understood that the base may be a form of a gene or genomic sequence relevant for diagnosing a disease or condition other than AML including, but not limited to, other cancers.

At step 704, a locus-specific error profile for an allele is received (e.g., by processor 110 of FIG. 1). It should be understood that a given allele may be comprised of multiple bases or a single base. For example, an allele is a form of a gene, part of a gene, or a non-coding genomic sequence; and a variant allele is a change in one or more bases with respect to either a reference sequence, or in the case of somatic variation, to a germline sequence. The locus-specific error profile for the allele can be obtained from a lookup table (LUT), which is stored in memory (e.g., memory 130 of FIG. 1). The address in the LUT can be generated based upon a locus where the allele is detected (see e.g., FIG. 3). As described herein, the LUT stores error profiles associated with respective loci in the genomic sequence. The locus-specific error profile is therefore associated with a location of the allele in the genomic sequence. The locus-specific error profile includes a threshold detection error rate. As described herein, the threshold detection error rates for specific loci in the genomic sequence are determined by experimentation (see e.g., FIG. 6). This may include a statistical analysis of the fidelity of data from a device that performs basecalling on sequences derived from specimens, the basecalling yielding basecalls and corresponding base-wise error scores. The locus-specific error profiles (which include threshold detection error rates) are then stored in the error profiles maintained in the LUT.

Additionally, as described herein, the LUT stores a plurality of sets of locus-specific error profiles for a given allele. It should be understood that the locus-specific error profile for the given allele depends on a number of factors, e.g., a sequencing device model, a basecaller algorithm, a kit type, a flowcell or chemistry type, or combinations thereof Through experimentation and statistical analysis, locus-specific error profiles can be determined for the given allele and combination of Q score threshold/library kit/input nucleic acid type/flowcell/basecaller, etc. The locus-specific error profile for an allele can therefore be retrieved using information about the sequencing device model, the basecaller algorithm, the kit type, the flowcell or chemistry type, or combinations thereof associated with the sequencing read that is received at step 702.

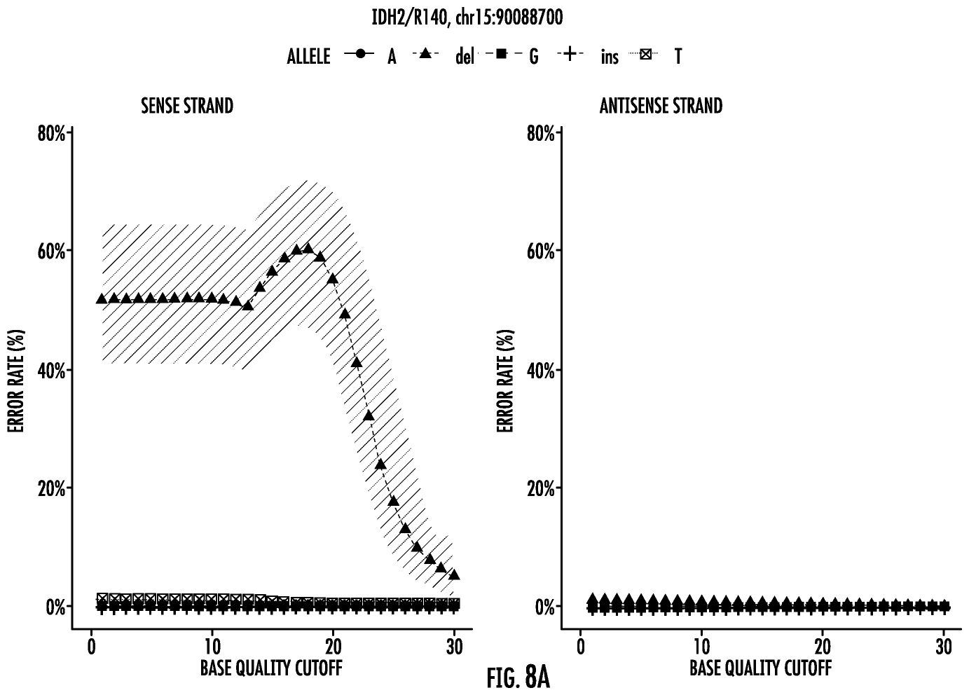

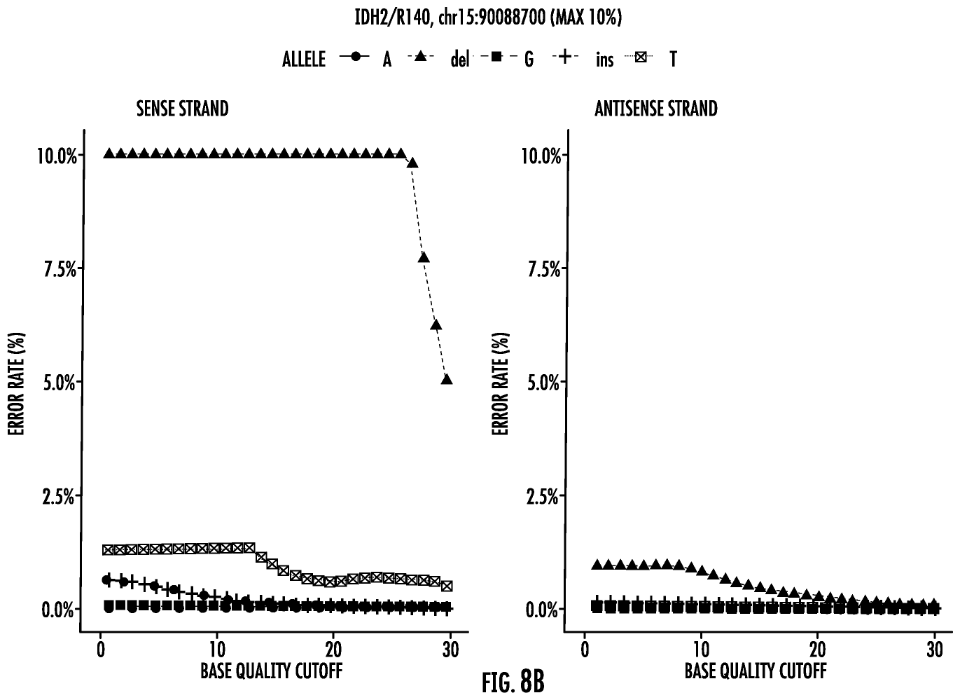

Alternatively or additionally, the locus-specific error profile is associated with a directionality of basecalling. It should be understood that directionality is dependent on which strand of the DNA duplex was sequenced and basecalled. For example, the “forward” direction is implicitly the top (“sense”) strand and the “reverse” direction is the bottom (“antisense”) strand, in relation to an implicitly double-stranded reference sequence. Additionally, four possible serializations of sequence may exist for a segment of duplex DNA: forward-top, reverse-bottom (reverse complement), reverse-top (reverse), and forward-bottom (complement). Directionality affects the electrical waveform output produced by the sequencing device due to a change in the nucleotide sequence being sampled by the device, and therefore also effects the error profiles. To see this, understand that even if the total reference sequence space spanned be the same, sequences originating from sense and anti-sense strands differ in both the serial order (forward versus reverse) of nucleotides as well as overall nucleotide composition (strands exhibit nucleobase pair-complementarity). Further, the effect on output signal is dependent on more than an individual nucleotide, as neighboring nucleotides within a window may influence the electrical field in a sampling pore or well. Thus, the directionality of basecalling (which strand—sense or antisense) results in different sequences and sequence contexts, which are associated with different error profiles. This is shown by FIGS. 8A and 8B. In other words, the locus-specific error profile for a given allele is not only dependent on local factors (e.g., Q score threshold/library kit/input nucleic acid type/flowcell/basecaller, etc.) but also dependent on directionality of basecalling. Therefore, locus-specific error profiles may contain one or more directionalities which may directly influence the error rates and error modes observed in the sequencing data. To see this, representative FIGS. 8A and 8B are provided; in each, the left panel represents error profiles on the sense strand, while the right panel represents error profiles of the antisense strand. In FIG. 8A, the complete figure is shown, whereas in FIG. 8B, the y-axis is capped at 10% to better visualize lower ranges. Overall, these figures showcase an example of the impact that the sequencing strand has on observed error rates and how awareness of the effect of strand can be utilized for mitigating errors present in the data by separately considering directional data. In this example, an erroneous allele present around in around 50% of sense-strand originating reads is only present at less than a 1% rate in the antisense reads, emphasizing the importance of allowing the profile to be built to separately assess strand-specific observations and alleles.

At 706, the base-wise error score associated with the base received at step 702 is compared to the threshold detection error rate for the base received at step 704.

At 708, the base is filtered based on the comparison (see e.g., FIG. 3). In particular, the base is either accepted as a true variant allele or discarded as a false positive allele based on the comparison. As described herein, the threshold detection error rate is associated with high confidence as to the veracity of variants observed in sequencing data (see e.g., FIG. 6). For example, the base is accepted as the true variant allele when the base-wise error score associated with the base is greater than or equal to the threshold detection error rate for the base. Or, the base is discarded as the false positive allele when the base-wise error score associated with the base is less than the threshold detection error rate for the base.

Optionally, after detecting a true variant allele according to steps 702-708 of FIG. 7, a patient is diagnosed with a disease or condition based upon the detection of the true variant allele. Thereafter, a therapy is delivered to the patient to treat the disease or condition.

It should be emphasized that the above-described embodiments of the present invention are merely possible examples of implementations, merely set forth for a clear understanding of the inventive principles and concepts. Many variations and modifications may be made to the above-described embodiments without departing substantially from the scope of the present disclosure. For example, the system 100 shown in FIG. 1 can have a variety of configurations. Likewise, many modifications can be made to the method described above with reference to FIGS. 2-8B without deviating from the scope of the present disclosure. All such modifications and variations are intended to be within the scope of this disclosure and the following claims.

1. A computer-implemented method for detecting alleles in a sample, the method comprising:
receiving a sequencing read, wherein the sequencing read comprises a basecall and a base-wise error score associated with a base within the sequencing read;
receiving a locus-specific error profile for an allele, wherein the locus-specific error profile comprises a threshold detection error rate;
comparing the base-wise error score associated with the base to the threshold detection error rate for the base; and
filtering the base based on the comparison, wherein the base is accepted as a true variant allele or discarded as a false positive allele based on the comparison.

2. The computer-implemented method of claim 1, wherein the base is accepted as the true variant allele when the base-wise error score associated with the base is greater than or equal to the threshold detection error rate for the base.

3. The computer-implemented method of claim 1, wherein the base is discarded as the false positive allele when the base-wise error score associated with the base is less than the threshold detection error rate for the base.

4. The computer-implemented method of claim 1, wherein the threshold detection error rate is associated with high confidence as to the veracity of variants observed in sequencing data.

5. The computer-implemented method of claim 1, wherein the step of receiving the locus-specific error profile for the allele further comprises reading the locus-specific error profile for the allele from a lookup table (LUT).

6. The computer-implemented method of claim 5, wherein the LUT stores a plurality of sets of locus-specific error profiles for the allele.

7. The computer-implemented method of claim 6, wherein each set of locus-specific error profiles for the allele is associated with a different combination of a sequencing device model, a basecaller algorithm, a kit type, and/or a flowcell or chemistry type.

8. (canceled)

9. The computer-implemented method of claim 1, wherein the locus-specific error profile is associated with a location of the allele in a reference genome.

10. The computer-implemented method of claim 9, wherein the locus-specific error profile is further associated with at least one of a sequencing device model, a basecaller algorithm, a kit type, or a flowcell or chemistry type.

11. The computer-implemented method of claim 10, further comprising receiving the at least one of the sequencing device model, the basecaller algorithm, the kit type, or the flowcell or chemistry type associated with the sequencing read.

12. The computer-implemented method of claim 1, wherein the locus-specific error profile is associated with a directionality of basecalling.

13. (canceled)

14. (canceled)

15. (canceled)

16. (canceled)

17. A method, comprising:
detecting a true variant allele according to the computer-implemented method of claim 1;
diagnosing a patient with a disease or condition based upon the detection of the true variant allele; and
delivering a therapy to the patient to treat the disease or condition.

18. The method of claim 17, wherein the disease or condition is Acute Myeloid Leukemia (AML).

19. A system for detecting alleles in a sample, the system comprising:
a processor; and
a memory in operable communication with the processor, wherein the memory has computer-executable instructions stored thereon that, when executed by the processor, cause the processor to:
receive a sequencing read, wherein the sequencing read comprises a basecall and a base-wise error score associated with a base within the sequencing read;
receive a locus-specific error profile for an allele, wherein the locus-specific error profile comprises a threshold detection error rate;
compare the base-wise error score associated with the base to the threshold detection error rate for the base; and
filter the base based on the comparison, wherein the allele is accepted as a true variant allele or discarded as a false positive allele based on the comparison.

20. The system of claim 19, further comprising a sequencing device configured to perform the sequencing read.

21. The system of claim 20, wherein the sequencing device is a next-generation sequencing (NGS) instrument.

22. The system of claim 19, wherein the base is accepted as the true variant allele when the base-wise error score associated with the base is greater than or equal to the threshold detection error rate for the base.

23. The system of claim 19, wherein the base is discarded as the false positive allele when the base-wise error score associated with the base is less than the threshold detection error rate for the base.

24. The system of claim 19, wherein the threshold detection error rate is associated with high confidence as to the veracity of variants observed in sequencing data.

25. The system of claim 19, wherein the step of receiving the locus-specific error profile for the allele further comprises reading the locus-specific error profile for the allele from a lookup table (LUT).

26. The system of claim 25, wherein the memory has further computer-executable instructions stored thereon that, when executed by the processor, cause the processor to maintain the LUT, wherein the LUT stores a plurality of sets of locus-specific error profiles for the allele.

27. The system of claim 26, wherein each set of locus-specific error profiles for the allele is associated with a combination of a sequencing device model, a basecaller algorithm, a kit type, and/or a flowcell or chemistry type.

28. (canceled)

29. The system of claim 19, wherein the locus-specific error profile is associated with a directionality of basecalling.

30-43. (canceled)

44. A system for detecting structural variants in a sample, the system comprising:
a processor in communication with the memory device and being configured to run a diagnostic tool, the processor receiving sequencing reads from a device when the device performs the basecalling algorithm to analyze the sample prepared using the kit, and wherein when the processor runs the diagnostic tool, the diagnostic tool performs a detection algorithm to determine whether an internal tandem duplication (ITD) of a gene is present in the sample.

45. The system of claim 44, wherein the device is a next-generation sequencing (NGS) instrument.

46. The system of claim 45, wherein the processor performs the detection algorithm by:
filtering for reads that meet the criterion of mapping to a locus of interest;
filtering for reads that meet the criterion of containing inserted sequence at or above a threshold length N;
constructing a distribution of insertion lengths;
heuristically selecting one or more peak lengths P={P1,P2, . . . Pn};
selecting from original filtered read set reads containing insertions within a preselected number of nucleotides (nt) of identified peaks P and grouping;
using a reference sequence, performing consensus calling with peak-specific read groups;
for each peak-specific group, updating the reference sequence to incorporate the consensus insertion; and
remapping the updated reference sequence to the original reference sequence to derive the final ITD(s).

47-49. (canceled)


SEQUENCE LISTING (3 sequences)

SEQ ID NO: 1 - DNA, 33 bp, Artificial Sequence
  Synthetic Construct
ttgagatcatattcatattctctgaaatcaacg

SEQ ID NO: 2 - DNA, 66 bp, Artificial Sequence
  Synthetic Construct
attcatattctctgaaatcaacgtagaagtactcattatctgaggagccggtcacctgtaccatcc

SEQ ID NO: 3 - DNA, 105 bp, Artificial Sequence
  Synthetic Construct
ttccattcttaccaaactctaaattttctcttggaaactcccatttgagatcatattcatattctctgaaatcaacgtagaagtactcattatctgaggagccgt